(19) United States
(12) Patent Application Publication
(10) Pub. No.: US 2007/0259043 A1
(43) Pub. Date: Nov. 8, 2007

(54) Halide-free glucosamine-therapeutic drug salt compositions

(75) Inventors: Vilas M. Chopdekar, Edison, NJ (US); Michael J. Torntore, Edison, NJ (US)

Correspondence Address: Law Offices of Jack Matalon, 32 Shelley Rd., Springfield, NJ, 07081-2529, US

(73) Assignee: Jame Fine Chemicals, Inc.

(21) Appl. No.: 11/223,686

(22) Filed: Sep. 9, 2005

Related U.S. Application Data
(60) Provisional application No. 60/611,178, filed on Sep. 17, 2004.

(51) Int. Cl. A61K 31/7008; A61K 9/14; C07H 5/04

(52) U.S. Cl. 424/486; 514/62; 536/18.7

(57) ABSTRACT

A salt of a glucosamine base having a purity of at least about 99 wt. % and a maximum halide content of about 0.01 wt. %, and a therapeutic drug having at least one acid functionality, e.g., a carbonyl moiety, a carboxyl moiety, a sulfoxide moiety, etc. Preferably, the salt is stabilized by coating it with at least one pharmaceutically acceptable polymer comprising a water-soluble, water-immiscible and/or water-swellable homopolymer and/or copolymer. Suitable polymers include carboxypolymethylene homopolymers and copolymers; polyethylene glycol homopolymers and copolymers, povidone homopolymers and copolymers; polyacrylic acid homopolymers and copolymers; polyacrylamide homopolymers and copolymers; polysaccharides; and mixtures of two or more of the foregoing polymers. The resultant coated halide-free glucosamine-therapeutic drug salt composition will be stable upon exposure to ambient temperature and/or the atmosphere. Suitable therapeutic drugs fall into the following classes: α- and β-Adrenergic Agonists; Narcotic and Non-Narcotic Analgesics; Anorexics; Antiallergics; Antianginals; Antiarrhythmics; Antiasthmatics; Antibiotics; Anticoagulants; Anticonvulsants; Antidepressants; Antidiabetics; Antihistaminics; Antihypertensives; Nonsteroidal Anti-Inflammatories; Antimigraines; Antineoplastics; Antiparkinsonians; Antipsychotics; Antipyretics; Antispasmodics; Antithrombotics; Antiulceratives; Anxiolytics; Decongestants; Diuretics; Hepatoprotectants; Sedatives; and Vasodilators.

### CROSS-REFERENCE TO RELATED APPLICATION

This application claims the benefit of Provisional Application Ser. No. 60/611,178 filed Sep. 17, 2004.

### FIELD OF THE INVENTION

The invention relates to halide-free glucosamine salts of therapeutic drugs having acid functionalities and to methods for preparing such salts.

### BACKGROUND OF THE INVENTION

Glucosamine is a well-known amino monosaccharide found in chitin, glycoproteins and glycosaminoglycans. Glucosamine is widely used for the treatment of rheumatic fever, arthritic and arthosic complaints, in the acute as well as chronic forms, as well as in the treatment of pathological conditions originating from metabolic disorders of the osteo-articular tissue. Although products in the marketplace are labeled as, or referred to as, “glucosamine”, they are misnomers since such products consist of glucosamine hydrochloride or as unreacted mixtures of glucosamine hydrochloride and a salt such as potassium or sodium sulfate.

One drawback of many therapeutic drugs is their relative insolubility in the body after they have been administered to a patient. It would be most desirable if more soluble versions of therapeutic drugs could be made available.

It has now been found that salts of a halide-free glucosamine base and therapeutic drugs having an acidic functionality are more soluble than the therapeutic drugs themselves. An added benefit is that glucosamine itself is formed in the body (typically in the form of glucosamine phosphate) and therefore no “foreign” ingredients will be introduced in the body when the salt compositions of the invention are administered to patients in need of such therapeutic drugs.

Salts or mixtures of “glucosamine” or “glucosamine sulfate” and a therapeutic drug such as aspirin, ibuprofen, ketoprofen are known in the prior art, e.g., see U.S. Patent Publication 2002/0058642 A1; U.S. Pat. No. 6,608,041 B2; U.S. Pat. No. 6,291,527 B1; U.S. Pat. No. 5,604,206; and U.S. Pat. No. 3,008,874. However, the “glucosamine” or “glucosamine sulfate” employed in such compositions are misnomers, inasmuch as such materials are actually glucosamine hydrochloride or mixed salts of glucosamine hydrochloride and an alkaline earth metal sulfate.

In contradistinction thereto, the glucosamine base employed in preparing the salts of the invention is halide free (i.e., the base has a purity of at least about 99 wt. % and a maximum halide content of about 0.01 wt. %) and as a result, the salts of the invention will contain neither a halide nor any extraneous anions nor any extraneous cations (e.g., sodium, potassium, calcium, etc.).

### DETAILS OF THE INVENTION

The starting materials for preparing the salts of the invention are a halide-free glucosamine base and a therapeutic drug having at least one acid functionality, e.g., a carbonyl moiety, a carboxyl moiety and/or a sulfoxide moiety. Glucosamine, extracted from shellfish or prepared by a fermentation process, is only available in the form of its hydrochloride salt. If the glucosamine hydrochloride salt is neutralized with a base, e.g., NaOH, KOH, etc. in order to release the glucosamine base, the resultant product will always contain a salt, i.e., NaCl or KCl, respectively, and it is not possible to separate the glucosamine base from the salt since both the glucosamine base and the salt are fully soluble in water.

Free glucosamine base may be prepared by the method recited in Chem. Ber., volume 75, page 1274. Such method involves the treatment of glucosamine hydrochloride with an ethanolic solution of a tertiary base such as triethylamine. Triethylamine hydrochloride is filtered off and the free glucosamine is then recovered from the reaction mixture. However, triethylamine is a toxic material even in small quantities and the yield of the free glucosamine is quite low. Moreover, the free glucosamine base still contains residual chloride.

A method for producing halide-free glucosamine base with a very high degree of purity has now been discovered. Such method is fully described in co-pending patent application Ser. No. ______, filed ______(corresponding to provisional application Ser. No. 60/611,709 filed Sep. 17, 2004). The aforesaid co-pending patent application is hereby incorporated in its entirety. By way of summary, the method disclosed in the aforesaid co-pending patent application is as follows:
(a) a glucosamine halide salt (e.g., glucosamine hydrochloride, glucosamine hydroiodide, etc.) is reacted with a lithium base in the presence of C₁-C₄ alcohol to thereby generate a C₁-C₄ alcohol solution of a lithium halide and an insoluble halide-free glucosamine base; and
(b) the insoluble halide-free glucosamine base is separated from the C₁-C₄ alcohol solution of the lithium halide salt.

For maximum yields, the reaction should be carried out at a temperature of about 15 to about 35° C.; conveniently, the reaction may be carried out at ambient temperatures. The C₁-C₄ alcohol may be, e.g., methanol, ethanol (preferably anhydrous), isopropanol, etc; the preferred alcohol comprises methanol. The lithium base may be anhydrous lithium hydroxide, lithium hydroxide monohydrate, lithium methoxide, lithium ethoxide or lithium isopropoxide. The preferred lithium base comprises anhydrous lithium hydroxide. It has been found that the presence of water in the reaction mixture reduces the yield of the halide-free glucosamine base. Accordingly, it is preferred that the reaction be carried out under anhydrous conditions. In general, the lithium base is employed in an amount of about 1.0 to about 1.2 moles per mole of halide present in the glucosamine halide salt. Excess lithium base is unnecessarily wasteful and will reduce the yield of the halide-free glucosamine base. Typically, the alcohol is employed in an amount of about 1 to about 10 parts, preferably 3 to 6 parts, per part of lithium base.

After allowing the reaction to proceed (preferably with stirring) for about 5 minutes to about 2 hours, the solid halide-free glucosamine base is filtered off from the resultant alcohol solution of the lithium halide and washed with additional alcohol. The halide-free glucosamine base may then be dried under vacuum at a temperature of about 15 to about 30° C. The yield typically ranges from about 85 to about 90%. The halide-free glucosamine base is quite pure. It will have a purity level of greater than about 99 wt. % and the halide content will be about 0.01 wt. % or less, e.g., 100 ppm or less and very often, the halide content will be less than 50 ppm and as low as 25 ppm. Based upon the residual halide content of the halide-free glucosamine base, the lithium residue in the glucosamine base will generally be about 20 ppm or less and very often, the lithium residue content will be less than 10 ppm.

The halide-free glucosamine base is quite hygroscopic and will decompose over a period of time if subjected to ambient temperature and/or to the atmosphere. Accordingly, it should be refrigerated in a closed container or preferably promptly used after recovery for conversion to the salts of the invention as described below.

The halide-free glucosamine base may be readily converted to the glucosamine base-therapeutic drug salt of the invention by reacting the base with a stoichiometric amount of a therapeutic drug having at least one acidic functionality. If the selected therapeutic drug has more than one acidic functionality, the molar ratio of the glucosamine base to the selected therapeutic drug should be adjusted such that there will be one mole of the glucosamine base employed per acidic functionality in the selected drug. Typically, the reaction mixture will comprise the glucosamine base, about 5 to about 30 parts, preferably 15 to 20 parts, of water (preferably purified water) per part of the base and the selected drug. Although lesser amounts of water may be employed, the resultant solutions may become too viscous to be properly agitated, particularly if the glucosamine base-therapeutic drug salt is not isolated from the reaction mixture, but is stabilized by the addition of a polymer to the reaction mixture, as described below. On the other, hand, excessive amounts of water may lead to reduced yields if a water-miscible solvent is used to recover the salt and if freeze-drying is used to recover the salt, the freeze-drying process becomes more time-consuming and expensive because of the large amount of water to be removed from the reaction mixture.

The selected therapeutic drug is slowly added to the aqueous solution of the glucosamine base while the aqueous solution is agitated, e.g. over a period of a few minutes, and the reaction mixture is further agitated for 5 to 120 minutes. The reaction is typically conducted at a temperature of about 15 to about 40° C.

Thereafter, the glucosamine base-therapeutic drug salt of the invention may be recovered from the reaction mixture by freeze-drying or by adding a water-miscible solvent such as acetone to the reaction mixture such that the salt will precipitate from the reaction mixture and the salt is then recovered by conventional filtration methods. The salt may then be dried by conventional methods, e.g., a stream of nitrogen, a vacuum oven at 30-50° C. for a period of 1 to 10 hours, etc. It is preferred that the recovery of the glucosamine base-therapeutic drug salt of the invention be carried out by a freeze-drying process as described in greater detail below.

Some of the glucosamine base-therapeutic drug salts of the invention may decompose over a period of time if they are exposed to ambient temperatures or the atmosphere. Therefore, it is preferred that the salt not be recovered from the reaction mixture as is, but converted to a stabilized form prior to recovery. Conversion of the salt to its stabilized form may be desirable even for those salts that do not decompose upon exposure to ambient temperatures and/or the atmosphere, since the pharmaceutically acceptable polymers employed in stabilizing, i.e., coating, the salts of the invention may provide extended-release properties when the salts are administered to warm-blooded vertebrates in need of treatment.

Stabilization of the glucosamine base-therapeutic drug salt is readily accomplished by adding a suitable pharmaceutically acceptable polymer to the reaction mixture prior to recovery of the salt. The pharmaceutically acceptable polymer may be a water-soluble, water-dispersible and/or or a water-swellable homopolymer and/or copolymer. Preferably the pharmaceutically acceptable polymer will be water-soluble. In general, the polymer will be employed in an amount of about 2 to about 70, preferably 20 to 50, parts by weight of the polymer per part of the salt in the reaction mixture.

Nonlimiting examples of commercially available pharmaceutically acceptable homopolymers and copolymers suitable for stabilizing the halide-free glucosamine-therapeutic drug salts of the invention include the following: carboxypolymethylene homopolymers and copolymers, i.e., vinyl polymers having active carboxyl groups such as high molecular weight homopolymers of acrylic acid crosslinked with allylsucrose or allylpentaerythritol and copolymers of acrylic acid modified by long chain C₁₀-C₃₀) alkyl acrylates and crosslinked with allylpentaerythritol—such polymers are commercially available and are marketed as Carbopol® polymers; polyethylene glycol homopolymers and copolymers (e.g., polyethylene-co-lactic acid copolymers); particularly polyethylene glycol polymers having molecular weights in the range of about 2,000 to about 20,000, preferably 4,000 to 18,000; polypropylene glycol homopolymers and copolymers, especially polypropylene glycol homopolymers having molecular weights of about 800 to about 18,000; ethylcellulose; povidone homopolymers, i.e., synthetic water-soluble homopolymers of N-vinyl-pyrrolidone, especially those having a molecular weight of about 2,500 to about 10,000; copovidone, i.e. synthetic random copolymers of N-vinylpyrrolidone and vinyl acetate in a 60:40 ratio; polyacrylic acid homopolymers and copolymers; polyacrylic homopolymers and copolymers; polysaccharides, etc.

The choice of particular homopolymers and/or copolymers for coating, i.e., stabilizing, the glucosamine base-therapeutic drug salt, is not critical so long as the polymers are pharmaceutically acceptable, have the capability of coating, i.e., stabilizing, the salt without any adverse chemical reaction occurring between the selected polymer and the salt and the resultant coated salt compositions are stable, i.e., they will not undergo decomposition when exposed to ambient temperatures and/or the atmosphere.

If the glucosamine-base therapeutic drug salt is to be recovered from the reaction mixture in a stabilized form, the desired pharmaceutically acceptable polymer is added, preferably in increments, with stirring, to the aqueous glucosamine base solution preferably prior to the addition of the therapeutic drug. This step will generally take about 5 to about 15 minutes and is preferably conducted at a temperature of about 15 to about 40° C. After all increments of the selected polymer have been added, stirring is continued for an additional 5 to 120 minutes. Thereafter, the organic acid is slowly added to the reaction mixture, while maintaining the reaction mixture at a temperature of about 15 to 40° C.

The last step is the recovery of the polymer-coated, i.e., stabilized, glucosamine base-therapeutic drug salt composition from the reaction mixture. The stabilized salt composition may be recovered from the reaction mixture by freeze-drying or by adding a water-miscible solvent, e.g., acetone, to the reaction mixture to cause the stabilized salt composition to precipitate out from the reaction mixture. The precipitate is then recovered by conventional filtration methods and it may be dried as described below. Of course, the choice of stabilizing polymer and water-miscible solvent should be such that the polymer will not dissolve in, or otherwise react with, the solvent.

The stabilized halide-free glucosamine-therapeutic drug salt composition of the invention is preferably recovered by removal of water from the reaction mixture by freeze-drying, a well-known technique for removing water from compositions. Although freeze-drying is a time-consuming process, (a reaction mixture containing one liter of water will typically require 30-36 hours to remove about 97% of the water), it is preferred since the formation of decomposition products resulting from heating the reaction mixture or adding solvents to the reaction mixture can be avoided.

The freeze-drying process will generally be carried out at a reduced pressure and reduced temperature, e.g., a pressure of not greater than 500 milliTorre, preferably 300 to 100 milliTorre and at a temperature of about −60 to about −20° C., preferably −50 to −40° C. The endpoint of the completion of the freeze-drying process may be determined by condensing and measuring the quantity of water removed during the freeze-drying process. The time required for completion of the freeze-drying process will vary depending on factors such as pressure, temperature, quantity of reaction mixture to be free-dried, level of water to be tolerated in the stabilized halide-free glucosamine-drug composition, the thickness and surface area of the reaction mixture in the trays of the freeze-drying equipment, etc.

If the stabilized glucosamine base-therapeutic drug salt composition is to be recovered by precipitation from the reaction mixture by addition of a water-miscible solvent such as acetone to the reaction mixture, generally about 2 to about 10 parts of solvent per part of reaction mixture will be required.

After the stabilized glucosamine base-therapeutic drug salt composition has been recovered from the reaction mixture, it may be dried by conventional techniques, e.g., a stream of nitrogen, vacuum oven at a temperature of about 30 to about 50° C. for 1 to 10 hours or more, etc.

It should also be noted that the stabilization of the glucosamine base-therapeutic drug salts of the invention may provide an additional advantage to warm-blooded vertebrates to whom such compositions are administered. The stabilized, i.e., polymer-coated, versions of the glucosamine-therapeutic drug salts may provide extended release properties, i.e., the glucosamine-therapeutic drug may be released within the vertebrate over an extended period of time, thereby possibly resulting in a reduction of the frequency and the amount of the dosage that would otherwise be required to be administered to the vertebrate.

The therapeutic drug that is to be saltified with the glucosamine base may be any therapeutic drug that exhibits an acidic pH, i.e., a pH of less than 7.0. Such drugs will contain one or more acidic functionalities such as carbonyl moiety, a carboxyl moiety, a sulfoxide moiety, etc. The list of therapeutic drugs that fit such definition is quite voluminous. Suitable therapeutic drugs containing at least one acidic functionality may be found in one or more of the following nonlimiting, representative classes of drugs; α- and β-Adrenergic Agonists; Narcotic and Non-Narcotic Analgesics; Anorexics; Antiallergics; Antianginals; Antiarrhythmics; Antiasthmatics; Antibiotics; Anti-coagulants; Anticonvulsants; Antidepressants; Antidiabetics; Antihistiminics; Antihypertensives; Nonsteroidal Anti-Inflammatories; Antimigraines; Antineoplastics; Antiparkinsonians; Antipsychotics; Antipyretics; Antispasmodics; Antithrombotics; Antiulceratives; Anxiolytics; Decongestants; Diuretics; Hepatoprotectants; Sedatives; and Vasodilators.

Not every possible therapeutic drug within the foregoing-listed classes will be suitable for preparing a salt with halide-free glucosamine base. Only those therapeutic drugs that are sufficiently acidic in nature to form such a salt with the glucosamine base are suitable. As mentioned above, such therapeutic drugs will have a pH of less than 7.0 and will contain at least one acid functionality, e.g. a carbonyl moiety, a carboxyl moiety, a sulfoxide moiety, etc.

Particularly suitable specific drugs within the foregoing classes include: acetaminophen, acetazolamide, ampicillin, ampiroxicam, aspirin, bromfenac, celecoxib, cetirizine, chlorothiazide, chloropropamide, ciprofloxacin, diclofenac, ethacrynic acid, flufenamic acid, furosemide, ibuprofen, indomethacin, indoprofen, ketoprofen, levodopa, meclofenamic acid, methotrexate, methyldopa, naproxen, orazamide, penicillamine, pentobarbital, phenobarbital, phenytoin, piroxicam, propylthiouracil, protoprophyrin IX, rofecoxib, salicyclic acid, sulfadiazine, sulfapyridine, sulindac, theophylline, thioctic acid, timonacin, tipronin, tolbutamide, tolfenamic acid, warfarin, tolmetin, zaltoprofen, and mixtures thereof, and the like.

The following nonlimiting examples shall serve to illustrate the preferred embodiments of the invention. Unless otherwise indicated, all parts and percentages are on a weight basis.

### EXAMPLE 1

A reaction vessel was equipped with a stirrer and a nitrogen blanket. To the reaction vessel were added 4.1 g (0.02 mole) of ibuprofen and 200 cc of pharmaceutical grade methanol. The mixture was stirred to obtain a solution and thereafter, 3.58 g (0.02 mole) of a halide-free glucosamine base were added to the reaction mixture. The reaction mixture was then stirred for 1 hour at 25-30° C., resulting in a clear solution. The methanol was stripped off from the reaction mixture using a rotary evaporator at a temperature of 50° C. The resultant glucosamine base-ibuprofen salt weighed 7 g.

### EXAMPLE 2

A reaction vessel was set up with a stirrer and a warm water bath. Into the reaction vessel were added 1.79 g (0.01 mole) of a halide-free glucosamine base and the mixture was stirred at 25-35° C. to obtain a clear solution. Thereafter, 3.57 g (0.01 mole) of indomethacin were added and the reaction mixture was stirred for 1 hour at 35-45° C. The reaction mixture was then freeze-dried at a pressure of about 200 milliTorre and a temperature of about −45° C. 3.8 g of a light yellow powder consisting of the glucosamine base-indomethacin salt were obtained.

### EXAMPLE 3

Example 2 was repeated using 8.6 g (0.05 mole) of halide-free glucosamine base, 150 cc of purified water and 7.54 g (0.05 mole) of acetaminophen. 15 g of a white powder consisting of the glucosamine base-acetaminophen salt were obtained.

### EXAMPLE 4

Example 2 was repeated using 9.0 g (slight excess above 0.05 mole) of halide-free glucosamine base, 150 cc of purified water and 9 g (0.05 mole) of acetylsalicyclic acid. 17.4 g of a white solid consisting of the glucosamine base-acetylsalicyclic acid salt were obtained.

### EXAMPLE 5

Example 2 was repeated using 1.79 g (0.01 mole) of halide-free glucosamine base, 100 cc of purified water and 2.3 g (0.01 mole) of naproxen. 3.8 g of a white product consisting of the glucosamine base-naproxen salt were obtained.

### EXAMPLE 6

Example 2 was repeated using 1.79 g (0.01 mole) of halide-free glucosamine base, 100 cc of purified water and 2.96 g (0.01 mole) of diclofenac. 4.0 g of an off-white powder consisting of the glucosamine base-diclofenac salt were obtained.

### EXAMPLE 7

Example 2 was repeated using 1.79 g (0.01 mole) of halide-free glucosamine base, 50 cc of purified water and 0.28 g (0.01 mole) of diazepam. 0.43 g of a white solid consisting of the glucosamine base-diazepam salt was obtained.

### EXAMPLE 8

Example 1 was repeated using 3.6 g (0.02 mole) of halide-free glucosamine base, 300 cc of pharmaceutical grade methanol and 5.04 g (0.02 mole) of phenytoin. 8 g (92% yield) of a white solid consisting of the glucosamine base-phenytoin salt were obtained.

What is claimed is:

1. A salt of a glucosamine base having a purity of at least about 99 wt. % and a maximum halide content of about 0.01 wt. %, and a therapeutic drug having at least one acid functionality.

2. The salt of claim 1 further comprising a pharmaceutically acceptable polymer.

3. The salt of claim 2 wherein the polymer comprises a water-soluble, water-dispersible and/or a water-swellable homopolymer and/or copolymer.

4. The salt of claim 2 wherein the polymer is selected from the group consisting of carboxypolymethylene homopolymers and copolymers; polyethylene glycol homopolymers and copolymers; polypropylene glycol homopolymers and copolymers; ethylcellulose; povidone homopolymers and copolymers; polyacrylic acid homopolymers and copolymers; polyacrylamide homopolymers and copolymers; polysaccharides; and mixtures of two or more of the foregoing polymers.

5. The salt of claim 2 wherein the polymer is present in an amount of about 2 to about 70 parts by weight, per part of the salt.

6. The salt of claim 1 wherein the therapeutic drug is selected from the group consisting of the classes of α- and β-Adrenergic Agonists; Narcotic and Non-Narcotic Analgesics; Anorexics; Antiallergics; Antianginals; Antiarrhythmics; Antiasthmatics; Antibiotics; Anticoagulants; Anticonvulsants; Antidepressants; Antidiabetics; Antihistaminics; Antihypertensives; Nonsteroidal Anti-Inflammatories; Antimigraines; Antineoplastics; Antiparkinsonians; Antipsychotics; Antipyretics; Antispasmodics; Antithrombotics; Antiulceratives; Anxiolytics; Decongestants; Diuretics; Hepatoprotectants; Sedatives; and Vasodilators.

7. The salt of claim 6 wherein the drug is selected from the group consisting of acetaminophen, acetazolamide, ampicillin, ampiroxican, aspirin, bromfenac, celecoxib, cetirizine, chlorothiazide, chlorpropamide, ciprofloxacin, diclofenac, ethacrynic acid, flufenamic acid, furosemide, ibuprofen, indomethacin, indoprofen, ketoprofen, levodopa, meclofenamic acid, methotrexate, methyldopa, naproxen, orazamide, penicillamine, pentobarbital, phenobarbital, phenytoin, piroxicam, propylthiouracil, protoprophyrin IX, rofecoxib, salicyclic acid, sulfadiazine, sulfapyridine, sulindac, theophylline, thioctic acid, timonacic, tiopronin, tolbutamide, tolfenamic acid, warfarin, tolmetin, zaltoprofen, and mixtures thereof.

8. A composition comprising a coated salt of a glucosamine base having a purity of at least about 99 wt. % and a maximum halide content of about 0.01 wt. %, and a therapeutic drug having at least one acid functionality, said coating comprising a pharmaceutically acceptable polymer such that the coated salt will be stable upon exposure to the atmosphere or ambient temperature.

9. The composition of claim 8 wherein the pharmaceutically acceptable polymer comprises a water-soluble, water-dispersible and/or a water-swellable homopolymer and/or copolymer.

10. The composition of claim 8 wherein the pharmaceutically acceptable polymer is selected from the group consisting of carboxypolymethylene homopolymers and copolymers; polyethylene glycol homopolymers and copolymers; polypropylene glycol homopolymers and copolymers; ethylcellulose; povidone homopolymers and copolymers; polyacrylic acid homopolymers and copolymers; polyacrylamide homopolymers and copolymers; polysaccharides; and mixtures of two or more of the foregoing polymers.

11. The composition of claim 8 wherein the pharmaceutically acceptable polymer is present in the composition in an amount of about 2 to about 70 parts by weight, per part of the salt.

12. The composition of claim 8 wherein the therapeutic drug is selected from the group consisting of the classes of α- and β-Adrenergic Agonists; Narcotic and Non-Narcotic Analgesics; Anorexics; Antiallergics; Antianginals; Antiarrhythmics; Antiasthmatics; Antibiotics; Anticoagulants; Anticonvulsants; Antidepressants; Antidiabetics; Antihistaminics; Antihypertensives; Nonsteroidal Anti-Inflammatories; Antimigraines; Antineoplastics; Antiparkinsonians; Antipsychotics; Antipyretics; Antispasmodics; Antithrombotics; Antiulceratives; Anxiolytics; Decongestants; Diuretics; Hepatoprotectants; Sedatives; and Vasodilators.

13. The composition of claim 12 wherein the drug is selected from the group consisting of acetaminophen, acetazolamide, ampicillin, ampiroxicam, aspirin, bromfenac, celecoxib, cetirizine, chlorothiazide, chlorpropamide, ciprofloxacin, diclofenac, ethacrynic acid, flufenamic acid, furosemide, ibuprofen, indomethacin, indoprofen, ketoprofen, levodopa, meclofenamic acid, methotrexate, methyldopa maproxen, orazamide, penicillamine, pentobarbital, phenobarbital, phenytoin, piroxicam, propylthiouracil, protoprophyrin IX, rofecoxib, salicyclic acid, sulfadiazine, sulfapyridine, sulindac, theophylline, thioctic acid, timonacic, tiopronin, tolbutamide, tolfenamic acid, warfarin, tolmetin, zaltoprofen, and mixtures thereof.

14. A method for preparing a salt of a glucosamine base having a purity of at least about 99 wt. % and a maximum halide content of about 0.01 wt. %, and a therapeutic drug containing at least one acid functionality comprising the steps of:
(a) dissolving the glucosamine base in water;
(b) adding a stoichiometric amount of the therapeutic drug to the aqueous solution resulting from step (a); and
(c) recovering the salt from the reaction mixture produced in step (b).

15. The method of claim 14 further comprising adding a pharmaceutically acceptable polymer to the reaction mixture resulting from step (a) prior to carrying out step (b) such that a stabilized salt composition is recovered in step (c).

16. The method of claim 15 wherein the pharmaceutically acceptable polymer comprises a water-soluble, water-dispersible and/or a water-swellable homopolymer and/or copolymer.

17. The method of claim 15 wherein the pharmaceutically acceptable polymer is selected from the group consisting of carboxypolymethylene homopolymers and copolymers; polyethylene glycol homopolymers and copolymers; polypropylene glycol homopolymers and copolymers; ethylcellulose; povidone homopolymers and copolymers; polyacrylic acid homopolymers and copolymers; polyacrylamide homopolymers and copolymers; polysaccharides; and mixtures of two or more of the foregoing polymers.

18. The method of claim 15 wherein the pharmaceutically acceptable polymer is added to the reaction mixture in an amount of about 2 to about 70 parts by weight, per part of the salt.

19. The method of claim 14 wherein step (c) is carried out by adding a water-miscible solvent to the reaction mixture so as to precipitate the salt therefrom.

20. The method of claim 18 wherein step (c) is carried out by freeze-drying.
